(19) United States
(12) Patent Application Publication
(10) Pub. No.: US 2009/0137905 A1
(43) Pub. Date: May 28, 2009

(54) ULTRASOUND PROBE

(76) Inventors: Malcolm John Watson, Glasgow (GB); George A. Corner, Glasgow (GB)

Correspondence Address: NIXON & VANDERHYE, PC, 901 NORTH GLEBE ROAD, 11TH FLOOR, ARLINGTON, VA, 22203, US

(21) Appl. No.: 11/915,942

(22) PCT Filed: May 31, 2006
(86) PCT No.: PCT/GB06/01975

§ 371 Date: Jul. 14, 2008

(30) Foreign Application Priority Data
May 31, 2005 (GB) 0511008.5

(51) Int. Cl. A61B 8/14

(52) U.S. Cl. 600/459

(57) ABSTRACT

An ultrasound probe for locating with respect to a surface of a target object. The ultrasound probe has a face suitable for receiving the palm of a hand of a user, allowing the user to accurately move, hold and/or grip the ultrasound probe using their hand. At least a portion of the face comprises an elastically deformable material.

The present invention relates to the field of ultrasonography and in particular, ultrasound probes.

Ultrasound probes are used in an array of medical examinations, diagnoses and applications. These include the detection of malignant and benign tumours, providing images of these for assessment of their development, monitoring blood flow within various vital organs and foetuses, and the identification of a lumbar interspace suitable for insertion of central neuroaxial (spinal, epidural or combined spinal epidural) anaesthesia. A variety of ultrasonigraphic techniques have been developed for such applications.

One of the most recent of these applications is that which facilitates the administration, by injection, of local anaesthetic into the sub-arachnoid or epidural region using an ultrasound probe to identify a suitable lumbar interspace.

The purpose of such an injection may be to provide analgesia to the patient. Alternatively, the anaesthetic may be administered to provide sufficient loss of sensation in the patient, to enable particular types of surgical procedures to be carried out. Such procedures might include: obstetric surgery, lower limb orthopaedic surgery, gynaecological surgery, general surgery, cardiothoracic surgery, and transplant surgery. This type of anaesthetic is referred to as a central neuroaxial block.

The use of ultrasound probes for the various applications referred to above can sometimes prove problematic. This is especially the case in the application of ultrasound probes in correctly identifying a safe lumber interspace into which an anaesthetic can be administered effectively. To carry out such a procedure, the user is required to accurately control and direct the ultrasound probe. Whilst the degree of control is important in all patient groups (and in all applications), it is particularly important in the case of obese patients, where excessive subcutaneous tissue prevents the palpation of subcutaneous landmarks, which can normally be exploited by a user as a guide.

The increased prevalence of morbid obesity in the British population presents a particular problem in obstetric and general anaesthetic practice. As mentioned, in obese patients the spinous processes are often impalpable, rendering the insertion of spinal, epidural and combined spinal epidural anaesthesia problematic.

Should the anaesthetist be unable to find an appropriate lumber interspace, or if the duration of multiple attempts threatens to adversely affect the outcome of the patient, then the usual alternative option for administering anaesthesia is the administration of a general anaesthetic to the patient. The use of a general anaesthetic will, particularly in the case of pregnancy, expose patients to a significantly increased risk of mortality and morbidity. This risk is increased further if the patient is obese.

Consequently, it is imperative that the user has full and accurate control of the ultrasound probe when administering a lumber anaesthetic to an obese patient. Also, it is important and desirable that a user has full control of an ultrasound probe when using it for other applications, (as discussed above) and on other patient groups.

Existing ultrasound probes have not been manufactured specifically for this use and, consequently, often prove difficult to use accurately and with comfort. In particular, when in use, many known ultrasound probes present the user with a flat surface which does not offer an easy point of grip. Also, ultrasound probes are presently made from hard, unyielding materials which, again, prove difficult to grip. Therefore, the degree of control that the user has over the ultrasound probe is often compromised. Also, the awkward gripping surface of known ultrasound probes can make their operation uncomfortable and tiresome for the user.

As known ultrasound probes are hard to grip and accurately control, it can be difficult for a user to maintain a good physical, and hence a good acoustic, contact between the ultrasound probe and the patient's back. Therefore, the quality and consistency of the images generated by ultrasound probes can be adversely affected by the inherent difficulty associated with gripping ultrasound probes.

A further consequence of ultrasound probes being hard to grip and control accurately is that it can be challenging to stabilise the transducers (which are located within the ultrasound probe housing). This is because it is difficult to brace an ultrasound probe against a patient's back during imaging. This can hinder needle insertion when an ultrasound probe is being used to identify a suitable area for administering anaesthesia.

Likewise, the inherent lack of grip and instability of existing ultrasound probes makes it difficult for a user to apply the device without introducing a degree of variability by angulation of the transducers relative to the sagittal plane. Consequently, traditional ultrasound probes make it difficult to image in the sagittal plane and thus often produce images that are over complicated and unhelpful.

The problems associated with known ultrasound probes are exacerbated by the use of acoustic gels and sterile sheaths. In use, ultrasound probes are often covered in such acoustic gels, before being encased in said sterile plastic sheaths. Under these circumstances, the user must try to grip the ultrasound probe through the sterile plastic sheath and the acoustic gel, thus further diminishing their ability to hold and accurately control the ultrasound probe.

It can be seen that encasing the ultrasound probe in an acoustic gel and a sterile plastic sheath compounds the problems associated with gripping and controlling the ultrasound probe. Again, this problem is of greatest significance when the ultrasound probe is being used to identify an area of the lumbar region suitable for the administration of anaesthesia by a needle.

The problems associated with known ultrasound probes can be exacerbated depending on the type of patient on which the equipment is being used. For example, a user may be required to operate an ultrasound probe for a prolonged period of time on an obese patient in attempting to assess the correct lumbar region for anaesthesia administration. This can be uncomfortable and laborious for the user, and can prove ineffective for the patient.

A further problem associated with existing ultrasound probes is that they are made from hard, rigid materials, and thus can be fragile and prone to damage. This is especially the case in labour wards, where the ultrasound probes are likely to be subject to heavy use and abuse. For example, when existing ultrasound probes are dropped, they are often damaged as the hard materials from which they are made offer little in the way of shock-absorption. Therefore, both the housing and the internal components of the ultrasound probe are easily broken.

Therefore it is an object of the present invention to obviate, or at least mitigate, at least some of the drawbacks associated with the prior art.

Further aims and objects of the invention will become apparent from reading the following description.

According to a first aspect of the present invention there is provided an ultrasound probe comprising an anterior face suitable for locating with respect to a surface of a target object, and a posterior face suitable for receiving a palm of a hand of a user, wherein at least a portion of the posterior face comprises at least one substantially elastically deformable material.

The substantially elastically deformable material can be malleable.

Preferably at least a portion of the posterior face comprises a substantially contoured surface.

At least a portion of the posterior face can comprise a substantially elastically deformable material contained within an external cover.

The anterior surface can take the form of a flat base.

Preferably the posterior face includes manipulation means, the manipulation means comprising the at least one elastically deformable material.

The manipulation means can comprise a substantially hemispherical section.

The substantially hemispherical section can comprise a front region located in proximity with a front surface of the ultrasound probe, and a rear region located in proximity with a rear surface of the ultrasound probe.

The substantially hemispherical section can also comprise a flat truncated lower surface and a contoured upper surface.

The flat truncated lower surface of the substantially hemispherical section can be attached to an upper surface of the anterior face.

Optionally the substantially hemispherical section comprises a substantially isosceles trapezoid shaped base which comprises two parallel sides and two non-parallel sides.

The parallel sides of the isosceles trapezoid base are substantially parallel with a front surface of the ultrasound probe.

Preferably the posterior face comprises a recess.

Preferably the recess is adapted to receive at least one digit of a hand of a user.

The recess can take the form of a central groove that runs substantially perpendicular to a front surface of the ultrasound probe.

Optionally the central groove runs from an edge of the isosceles trapezoid base closest to the front surface of the ultrasound probe, towards the middle of the substantially hemispherical section.

The central groove can run towards the middle of the substantially hemispherical section in an upward manner with respect to the anterior face.

Optionally the substantially hemispherical section comprises side indentations that run from the middle of the non-parallel sides of the isosceles trapezoid base toward the rear region of the hemispherical section.

The side indentations can run towards the rear region of the hemispherical section in an upward manner with respect to the anterior face.

Optionally the hemispherical section forms a hollow on top of an upper surface of the anterior face.

Optionally the hollow comprises a first material, and the hemispherical section comprises a second material.

Alternatively the hollow comprises a sac.

The sac may comprise a first material and the hemispherical section may comprise a second material.

At least one of the first and second materials can be elastically deformable.

The first and second materials can be different materials.

Preferably the posterior face yields to the shape of the palm of a user's hand.

Preferably the target object is a human body.

It will now be described, by way of example only, various embodiments of the invention with reference to the following drawings of which;

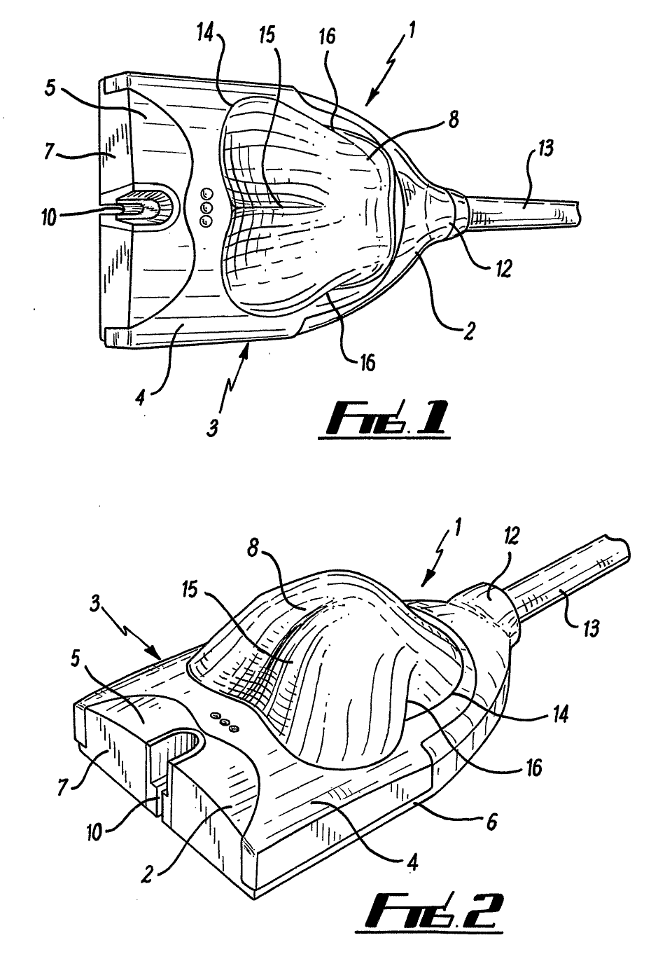

FIG. 1 shows a plan view of the ultrasound probe;

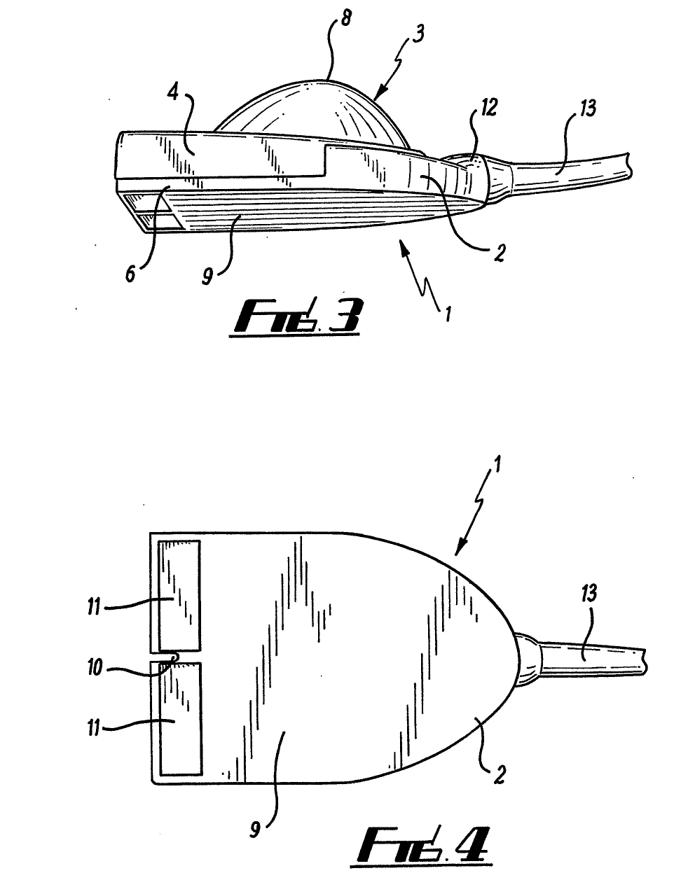

FIG. 2 shows a perspective view of the ultrasound probe;

FIG. 3 shows a side view of the ultrasound probe;

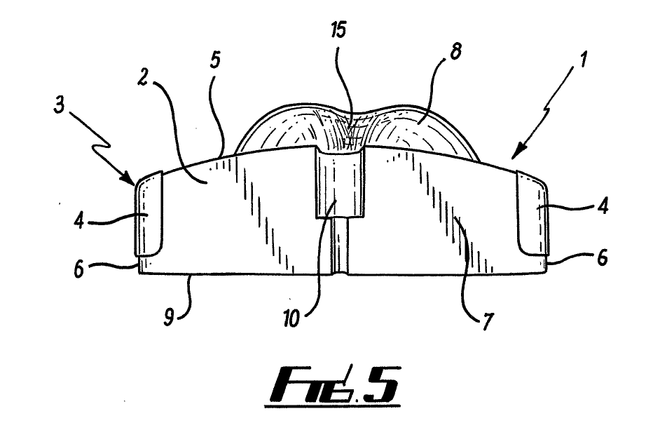

FIG. 4 shows a bottom view of the ultrasound probe; and

FIG. 5 shows a front view of the ultrasound probe.

Referring to FIGS. 1 and 2, an ultrasound probe 1 has a housing 2, having an anterior face 9, which in use is located on a surface of the target object on which the probe 1 is being used, and a posterior face 3, opposite the anterior face 9.

The posterior face is formed from a malleable, soft, elastically deformable material and has improved ergonomics over existing probes. The anterior face acts as a base, whilst the posterior face is padded or elevated such that it is adapted to receive the palm of the user. This provides a user with manipulation means. Manipulation means is understood to be a device suitable for moving, holding and/or gripping the ultrasound probe using a hand.

The whole of the posterior face may be formed from the malleable, soft, elastically deformable material which forms the manipulation means. Alternatively, the posterior face may include additional manipulation means. The additional manipulation means can be a raised portion (raised from the posterior face), the raised portion being formed from the malleable, soft, elastically deformable material.

It will be appreciated that as the posterior face is formed from a malleable, soft material it will have superior comfort and the capacity to mould to the shape of the user's hand. The posterior face has a substantially curvilinear or contoured surface configured to receive the palm of a user, in which there is located a recess, groove or channel. The recess is adapted to receive a digit of the user, typically the forefinger. It will be appreciated that in use this facilitates fine movement of the probe.

Surprisingly it has been found that the soft elastically deformable material provides the user with a greater degree of control than would be expected. Furthermore, it affords the user fine precision control not previously available with ultrasound probes. This is somewhat counter-intuitive as one might expect that a hard non-deformable handle, which can be more tightly gripped, would afford greatest control to a user. The present invention illustrates that in fact an elastically deformable grip provides a user with a higher degree of control than a hard non-deformable grip.

The ultrasound probe may be of an integral one piece construction or alternatively the posterior face may be of a separate construction to the anterior face.

The posterior face 3 has a hemispherical section 8 that protrudes in an upward manner from an upper surface 5 of the housing 2. At the rear of the housing 2 there is a socket 12 which can accept a cable 13.

In this embodiment, the malleable material 3 comprises a skirt 4 around the probe 1 which has the additional effect of protecting the housing 2 and its internal components (not shown), from being physically damaged. Specifically, the posterior face 3 is made from a pliable material and functions as a shock absorber, therefore protecting the housing 2 that it covers.

As ultrasound probes are often used in labour wards, they are likely to be subject to heavy use and abuse. The posterior face offers protection to the ultrasound probe in this type of environment. The posterior face makes the ultrasound probe more durable and abuse resistant.

Still referring to FIGS. 1 and 2, the hemispherical section 8 is ergonomically shaped such that a user (not shown) has complete control over the ultrasound probe 1 and can use the ultrasound probe 1 with a better degree of comfort.

In this embodiment the hemispherical section 8 has a substantially isosceles trapezoid shaped base 14, the parallel sides of the isosceles trapezoid base 14 being parallel with a front surface 7 of the housing 2. It will be appreciated, however, that the base shape 14 is not an exact isosceles trapezoid, nor is it intended to be limited to an exact isosceles trapezoid. Rather, the base shape 14, when viewed from above, appears as an approximate isosceles trapezoid.

In this embodiment, the ergonomically shaped hemispherical section 8 comprises a frontal indentation (or central groove) 15 that runs parallel to lateral surfaces 6, and from the edge of the isosceles trapezoid base 14 closest to the front surface 7, towards substantially the middle of the hemispherical section 8, in an upward manner.

The ergonomically shaped hemispherical section 8 comprises side indentations 16 that run from substantially the middle of the non-parallel sides of the isosceles trapezoid base 14 toward the rear of the hemispherical section 8, in an upward manner.

These side and front indentations prevent the isosceles trapezoid shaped base from being an exact isosceles trapezoid.

The ergonomic shape described in detail herein provides the user with a comfortable and controllable ultrasound probe. However, it will be appreciated that any suitable ergonomic shape which facilitates an increased degree of control and comfort for the user can be employed.

Referring now to FIG. 3, the posterior face 3 comprises a separate component (in other words is not integral to the anterior face) attached to the upper surface (not shown) of the housing 2 of the ultrasound probe 1. In this illustration it can be seen that the posterior face 3 has a raised section that resembles a hemisphere (the hemispherical section 8), which is attached to the housing 2 by the flat (or truncated) surface of the hemispherical section (not shown). In this representation, the anterior face 9 and the lateral surfaces 6 can also be seen. In addition, the skirt 4, socket 12 and cable 13 are evident.

Referring now to FIG. 4, again the ultrasound probe is depicted at 1. The anterior face 9 of the housing 2 can be seen, as can two transducers 11, which are contained within the housing 2, and a guide gap 10. Also visible is the cable 13.

In this example, the guide gap is a needle guide for use in directing a needle being used in anaesthesia. However, it will be appreciated that any suitable guide means, and any suitable needle guide, may be used.

Referring to FIGS. 1 and 4, the lateral surfaces 6 of the housing 2 are substantially parallel where they join the front surface 7 and taper towards each other as they approach the rear of the housing 2 such that the housing 2, when viewed from above the upper surface 5 or below the anterior face 9 appears bell-shaped. At the rear of the housing 2 the lateral surfaces 6 marry and are shaped to form a socket 12 which can accept a cable 13.

Referring now to FIG. 5, there is again shown the ultrasound probe 1, and it can be seen that the guide gap 10 extends from the upper surface 5 to the anterior face 9 and in doing so forms a channel in the front surface 7. Also, the contours of the hemispherical section 8 of the posterior face 3 are apparent in this illustration. In particular, it can be seen that the frontal indentation 15 that runs parallel to the lateral surfaces 6 forms a “B” shape, when viewed from this perspective. This diagram also shows the skirt 4, partially covering the front surface 7.

In the embodiments described the posterior face is made from a pliable material. The hemispherical section and the skirt are both made from the same pliable material.

In an alternative embodiment, the hemispherical section forms a hollow on top of the upper surface of the housing, the hollow being filled with a first material, and the hemispherical section being made from a second material. Generally the first and second materials are different.

In a further alternative embodiment, the hemispherical section forms a hollow on top of the upper surface of the housing, the hollow being filled with a sac, the sac containing a first material and the hemispherical section being made from a second material. Generally the first and second materials are different.

In these alternative embodiments, the hemispherical section can extend into the skirt to form an external cover over the posterior face, the external cover encapsulating a pliable material, or a sac containing a pliable material.

In use, the ergonomically shaped posterior face allows the user to hold the probe in the palm of one hand with the ergonomic hemispherical section filling the whole of the palm of the hand. This, combined with the frontal indentation into which the index finger fits, allows the user to press the probe against a patient's back, and thus gain a good acoustic contact. When in use, the index finger points to the guide gap, in the same manner that an index finger might point when using a computer mouse.

In contrast to known ultrasound probes, the ultrasound probe with improved posterior face allows the user to securely grip the probe even when the probe is covered in acoustic gel and encased in a sheath. Therefore a good physical, and thus good acoustic, contact is maintained with the patient's back. Consequently, the quality and consistency of the image generated by the ultrasound probe is greatly enhanced.

The ease of use and controllability of the ultrasound probe with improved posterior face means that it is easier to stabilise the transducers (which are located within the ultrasound probe housing). This is a result of the user's ability to better brace the ultrasound probe against a patient's back during imaging. This can aid needle insertion when the ultrasound probe is being used to administer anaesthesia.

Similarly, the increased grip and stability of the ultrasound probe with improved posterior face makes it easier for a user to apply the device without adding a degree of variability, which is introduced by angulation of the transducers relative to the sagittal plane. Consequently, accurate imaging in the sagittal plane is more achievable, producing images that are less complex and more accurate.

As alluded to already, the problems associated with known ultrasound probes can be exacerbated depending on the type of patient on which the equipment is being used. For example, a user is often required to operate an ultrasound probe for a prolonged period of time on an obese patient in attempting to assess the correct lumbar region for anaesthesia administration. This can be painful and laborious for the user, and can prove ineffective for the patient. The increased degree of control and comfort that is presented by the ultrasound probe with improved posterior face means that it is quicker, easier and less painful for the user to carry out such a procedure. Moreover, it is also quicker and more effective for the patient.

The ultrasound probe with improved posterior face also serves to protect the internal components of the ultrasound probe. The soft, pliable elastically deformable material from which it is made offers a shock-absorbing, physically protective layer which shields against damage from heavy use and abuse. This is in contrast to existing ultrasound probes which do not have any type of physical protection and which are fragile and prone to damage.

Whilst the elastically deformable material of the present example is used on a bell-shaped housing, it will be appreciated that the elastically deformable material can be adapted such that it is suitable for attachment to any shape of ultrasound probe. Furthermore, it will be understood that the elastically deformable material need not necessarily be limited to use with an ultrasound probe, but will also be compatible with an array of hand held devices, medical or otherwise.

Surprisingly it has been found that the soft elastically deformable material provides the user with a degree of fine precision control, which was not previously available with ultrasound probes. This is somewhat counter-intuitive as one might expect that a hard non-deformable handle, which can be more tightly gripped, would afford greatest control to a user. The present invention illustrates that in fact an elastically deformable grip provides a user with a higher degree of control than a hard non-deformable grip.

Further modifications may be made without departing from the scope of the invention herein intended.

1. An ultrasound probe comprising an anterior face suitable for locating with respect to a surface of a target object, and a posterior face suitable for receiving a palm of a hand of a user, wherein at least a portion of the posterior face comprises at least one substantially elastically deformable material.

2. An ultrasound probe as claimed in claim 1, wherein the substantially elastically deformable material is malleable.

3. An ultrasound probe as claimed in claim 1, wherein at least a portion of the posterior face comprises a substantially contoured surface.

4. An ultrasound probe as claimed in claim 1, wherein at least a portion of the posterior face is comprised of a substantially elastically deformable material contained within an external cover.

5. An ultrasound probe as claimed in claim 1, Wherein the anterior surface takes the form of a flat base.

6. An ultrasound probe as claimed in claim 1, wherein the posterior face includes manipulation means, the manipulation means comprising the at least one elastically deformable material.

7. An ultrasound probe as claimed in claim 6, wherein the manipulation means comprises a substantially hemispherical section.

8. An ultrasound probe as claimed in claim 7 wherein the substantially hemispherical section comprises a front region located in proximity with a front surface of the ultrasound probe, and a rear region located in proximity with a rear surface of the ultrasound probe.

9. An ultrasound probe as claimed in claim 8 wherein the substantially hemispherical section comprises a flat truncated lower surface and a contoured upper surface.

10. An ultrasound probe as claimed in claim 9 wherein the flat truncated lower surface of the substantially hemispherical section is attached to an upper surface of the anterior face.

11. An ultrasound probe as claimed in claim 7, wherein the substantially hemispherical section comprises a substantially isosceles trapezoid shaped base which comprises two parallel sides and two non-parallel sides.

12. An ultrasound probe as claimed in claim 11 wherein the parallel sides of the isosceles trapezoid base are substantially parallel with a front surface of the ultrasound probe.

13. An ultrasound probe as claimed in claim 1, wherein the posterior face comprises a recess.

14. An ultrasound probe as claimed in claim 13 wherein the recess is adapted to receive at least one digit of a hand of a user.

15. An ultrasound probe as claimed in claim 13, wherein the recess takes the form of a central groove that runs substantially perpendicular to a front surface of the ultrasound probe.

16. An ultrasound probe as claimed in claim 15, wherein the central groove runs from an edge of the isosceles trapezoid base closest to the front surface of the ultrasound probe, towards the middle of the substantially hemispherical section.

17. An ultrasound probe as claimed in claim 16, wherein the central groove runs towards the middle of the substantially hemispherical section in an upward manner with respect to the anterior face.

18. An ultrasound probe as claimed in claim 7, wherein the substantially hemispherical section comprises side indentations that run from the middle of the non-parallel sides of the isosceles trapezoid base toward the rear region of the hemispherical section.

19. An ultrasound probe as claimed in claim 18 wherein the side indentations run towards the rear region of the hemispherical section in an upward manner with respect to the anterior face.

20. An ultrasound probe as claimed in claim 7 wherein the hemispherical section forms a hollow on top of an upper surface of the anterior face.

21. An ultrasound probe as claimed in claim 20 wherein the hollow comprises a first material, and the hemispherical section comprises a second material.

22. An ultrasound probe as claimed in claim 20, wherein the hollow comprises a sac.

23. An ultrasound probe as claimed in claim 22 wherein the sac comprises a first material and the hemispherical section comprises a second material.

24. An ultrasound probe as claimed in claim 20, wherein at least one of the first and second materials is elastically deformable.

25. An ultrasound probe as claimed in claim 20, wherein the first and second materials are different materials.

26. An ultrasound probe as claimed in claim 1, wherein the posterior face yields to the shape of the palm of a user's hand.

27. An ultrasound probe as claimed in claim 1, wherein the target object is a human body.
